(19) United States
(12) Patent Application Publication
(10) Pub. No.: US 2022/0256784 A1
(43) Pub. Date: Aug. 18, 2022

(54) QUANTITATIVE WEED MONITORING METHOD

(71) Applicant: NANJING AGRICULTURAL UNIVERSITY, Nanjing, Jiangsu (CN)

(72) Inventors: Sheng QIANG, Nanjing (CN); Zheng ZHANG, Nanjing (CN)

(73) Assignee: NANJING AGRICULTURAL UNIVERSITY, Nanjing, Jiangsu (CN)

(21) Appl. No.: 17/616,363

(22) PCT Filed: Jun. 3, 2020
(86) PCT No.: PCT/CN2020/094114

§ 371 Date: Dec. 3, 2021

(30) Foreign Application Priority Data
Jun. 5, 2019 (CN) 201910485266.2

(51) Int. Cl. A01G 22/22

(52) U.S. Cl. CPC A01G 22/22

(57) ABSTRACT

A quantitative weed monitoring method guides the selection of weed prevention and control measures, and belongs to the field of plant protection. The method performs rapid and quantitative monitoring of weeds in a farmland in different farming modes in the whole growth period by using a four-level visual observation method in the seedling stage, a sampling method in the growth stage, a seven-level visual observation method in the mature stage, and a soil seed bank survey before planting, and implements a targeted weed control policy according to a quantitative index of the weeds, thereby achieving continuous prevention and control of the weeds. Through continuous monitoring, the occurrence rule and dynamic of the weeds can be determined, the weed prevention and control measures are implemented, and the usage frequency and amount of herbicides are reduced, so that the weed occurrence is controlled, and the planting costs and environmental pollution are reduced.

### TECHNICAL FIELD

The present invention relates to the field of crop cultivation and management technologies, and in particular, to a quantitative weed monitoring method.

### BACKGROUND

Weeds are one of the most important factors leading to reduction of crop yields. Weed control mainly depends on chemical herbicides. With the intensive and scale development of agricultural production in China, this dependence will be aggravated. In a rice-wheat (or rape) continuous farming region, herbicides can be used thrice for rice of one crop season, with “first for sealing”, “second for killing”, and “third for supplementing”, and herbicides can be used twice for wheat or rape, with “first for sealing” and “second for supplementing”. If a weed base is quite high, herbicides that kill poaceae weeds and broadleaf weeds are separately used in the “second for supplementing”, up to six times in total. However, long-term and large-scale application of chemical herbicides may cause serious pollution on farmland soil and water sources, serious environmental crisis, and excessive chemical residues in agricultural products. Some residues are environmental hormones that endanger human health. Another technical problem is generation of resistant weeds, which reduces the control effect and increases the usage amount of herbicides, thereby causing a vicious circle. Ultimately, the weeding costs are increased and the benefits of growers are reduced.

As people pay more attention to ecological issues such as food safety and environmental degradation, the weed control technology needs to change a current situation of almost completely depending on the chemical weeding technology. The Ministry of Agriculture also proposed a “double reduction” goal of reducing the use of chemical pesticides and fertilizers. The use of herbicides has accounted for more than 40% of the use of pesticides. Therefore, reducing the use of herbicides is an important part of achieving the “double reduction” goal. How to scientifically formulate weed control measures and reduce the usage frequency/amount of herbicides requires quantitative weed monitoring. Based on monitoring survey results, ecological weed control measures and chemical weeding measures are organically combined, and a comprehensive weed control technology is developed based on the quantitative weed monitoring, thereby finally implementing sustainable management of weeds.

An ecological weeding method of CN 103477920 B discloses an ecological weed control system research based on accelerating depletion of a weed seed bank. An ecological weed control technology applies irrigation water flow for filtration and removes floating weed seeds during irrigation and soaking stages, thereby resulting in a good control effect on weeds in a rice-wheat continuous farming field, and continuously decreasing the density of weed seeds in the soil seed bank year by year. In a farmland with a high weed density, use of ecological weed control measures is not enough to control occurrence and hazard of weeds in a current season, and chemical herbicides are also required. However, for how to perform rapid quantitative weed monitoring in cultivation and farming modes such as rice-wheat, rice-rape, rice-green manure, and rice-winter fallow field, and select a comprehensive weed control policy based on quantitative monitoring, to rapidly reduce the weed density and reduce the usage frequency and usage amount of herbicides, there is no relevant specific technology. Particularly, for how to rapidly determine a dominance level (or a hazard level) of the weeds according to abundance and coverage of the weeds in the field, there are no clear data indexes as a basis.

### SUMMARY

An objective of the present invention is to resolve a time-consuming and complex farmland weed survey method, and provide a quantitative weed monitoring method, which can perform rapid and quantitative monitoring and survey of weeds to determine the degree of weed hazard and provide a basis for the selection of a weed prevention and control policy.

The present invention is achieved by the following technical solutions:

A quantitative weed monitoring method includes the following steps:

(1) during a weed survey in the seedling stage, observing abundance and coverage of weeds in the seedling stage using a four-level visual observation method, and determining a dominance level of the weeds, where the four-level visual observation method is specifically as follows:

level one: observing that soil is obviously covered with green when standing straightly on a position of three meters away from a field, indicating high density occurrence of the weeds;

level two: observing that the soil is covered with green when standing straightly on an edge of the field or in the field, indicating medium density occurrence of the weeds;

level three: observing weed seedlings when bending down in the field, indicating low density occurrence of the weeds; and

level four: observing sporadic weed seedlings after squatting down in the field, indicating extremely low density occurrence of the weeds;

(2) during a weed survey in the mature stage, observing a height, abundance, and coverage of weeds relative to crops using a seven-level visual observation method, and determining a dominance level of the weeds, where the seven-level visual observation method is named in descending order of dominance levels of the weeds:

level one and level two: dominance levels 4 and 5, indicating serious weed hazard;

level three and level four: dominance levels 2 and 3, indicating medium weed hazard;

level five: a dominance level 1, indicating light weed hazard; and

level six and level seven: dominance levels 0 and T, indicating extremely light weed hazard;

(3) during a weed survey in the growth stage, using a “W”-shaped nine-point quadrat method, counting types of weeds and a quantity of plants of each type of weed in each of 9 quadrats with an area of 0.5-1 m² and designed in each field according to a “W” shape, and calculating a weed density; and

(4) after crop harvest, using a soil seed bank to drill the soil for sampling: 9 sampling points are designed in each field according to the “W” shape, and 5 points are set around the sampling points with an interval of one meter according to a shape of “plum blossom”; one soil sample is drilled at each point, a drilling depth is 15 cm, a drilling area of each soil sample is 0.002 m², and a drilling diameter is 50 mm; and after the soil sample is treated by a water washing method, types and quantities of weed seeds in the seed bank are counted as a soil weed seed bank scale.

Preferably, when the monitoring survey result is the high density occurrence of the weeds, the dominance levels 4 to 5, the weed density of >200 plants/m², or a soil weed seed bank scale of >100000 grains/m², during crop planting, a local conventional chemical weeding manner is used, in cooperation with ecological weed control measures “damming” and “net fishing”.

Preferably, when the monitoring survey result is the medium density occurrence of the weeds, the dominance levels 2 to 3, the weed density of 100-200 plants/m², or a soil weed seed bank scale of 50000-100000 grains/m², during crop planting, use of chemical herbicides is reduced once compared with the local conventional chemical weeding manner, in cooperation with ecological weed control measures “damming” and “net fishing”.

Preferably, when the monitoring survey result is the low density occurrence of the weeds, the dominance level 1, the weed density of 50-100 plants/m², or a soil weed seed bank scale of 20000-50000 grains/m², during crop planting, chemical herbicides are only used once, in cooperation with ecological weed control measures “damming” and “net fishing”.

Preferably, when the monitoring survey result is the extremely low density occurrence of the weeds, the dominance levels 0-T, the weed density of <50 plants/m², or a soil weed seed bank scale of <20000 grains/m², during crop planting, no chemical herbicides are used, and ecological weed control measures “damming” and “net fishing” are only used to maintain the weed density in the field at a low level.

Preferably, the farming mode of the crops is rice-wheat, rice-rape, rice-green manure, or rice-winter fallow field.

Beneficial effects of the present invention are as follows:

In the present invention, rapid and quantitative monitoring of weeds in a farmland in different farming modes in the whole growth period is performed by using a four-level visual observation method in the seedling stage, a sampling method in the growth stage, a seven-level visual observation method in the mature stage, and a soil seed bank survey before planting, and a targeted weed control policy is implemented according to a quantitative index of the weeds, thereby achieving continuous prevention and control of the weeds. Through continuous monitoring, the occurrence rule and dynamic of the weeds can be determined, the weed prevention and control measures are properly implemented, the usage frequency and usage amount of herbicides are reduced, so that the quantification of weed prevention is implemented, the weed occurrence is effectively controlled, planting costs are significantly reduced, and environmental pollution is reduced.

### DETAILED DESCRIPTION

The present invention is described in further detail below with reference to specific embodiments.

A quantitative weed monitoring method is a method of performing rapid and quantitative monitoring on weed occurrence in a farmland in different farming modes by using visual methods, and is applied to guide the proper selection of weed prevention and control measures. The method includes the following steps:

(1) During a weed survey in the seedling stage, the occurrence degree (or occurrence) of weeds is evaluated using a four-level visual observation method and a dominance level (or a hazard level) of the weeds is determined mainly according to green coverage of the soil surface. Different soil weed seed bank scales determine the occurrence amount of the weeds. During the weed survey in the seedling stage, the surveyor determines an occurrence situation of the weeds by changing an observation angle. The four-level visual observation method is specifically as follows: 1. observing when standing straightly: if it is observed at a position (three meters away from a field) that soil is obviously covered with green (a field of green), the weeds are of high density; 2. observing when standing straightly in the field: if the soil is covered with green, the weeds are of medium density; 3. observing when bending down in the field: if weed seedlings are observed, the weeds are of low density; and 4. observing after squatting down: if sporadic weed seedlings are observed, the weeds are of very low density. Refer to Table 1 for specific grading.

TABLE 1
Grading standard of four-level visual observation method for weeds in seedling stage

| Weed occurrence | Observation manner | Weed occurrence situation |
|---|---|---|
| High density | Observe when standing straightly | Soil is obviously covered |
| | at a position three meters away | with green |
| | from the field | |
| Medium density | Observe when standing straightly | Soil is covered with green |
| | in the field | |
| Low density | Observe when bending down in the | Weed seedlings are observed |
| | field | |
| Extremely low density | Observe after squatting down | Sporadic weed seedlings are |
| | | observed |

(2) During a weed survey in the mature stage, observing a height, abundance, and coverage of weeds relative to crops are observed using a seven-level visual observation method, and a dominance level (or a hazard level) of the weeds is determined. Hazard levels of weeds in a descending order are sequentially 5, 4, 3, 2, 1, T, 0, a total of 7 levels. The height of the weeds relative to the crops may be divided into three types: an upper level, a medium level, and a lower level. Relative heights of different weeds need to respectively correspond to different coverage and abundance, to determine grades of the hazard levels. Refer to Table 2 for specific grading.

TABLE 2
Grading standard of seven-level visual observation
method for dominance of weed communities

| Weed | Dominance level | | Relative | | Relative |
|---|---|---|---|---|---|
| density | (hazard level) | Assignment | coverage (%) | Abundance | height |
| Extremely | 5 | 5 | >25 | Many to quite | Upper level |
| high or high | | | | many | |
| density | | | >50 | Quite many | Medium level |
| | | | >95 | Quite many | Lower level |
| | 4 | 4 | 10-25 | Relatively | Upper level |
| | | | | many | |
| | | | 25-50 | Many | Medium level |
| | | | 50-95 | Quite many | Lower level |
| Medium | 3 | 3 | 5-10 | Relatively few | Upper level |
| density | | | 10-25 | Relatively | Medium level |
| | | | | many | |
| | | | 25-50 | Many | Lower level |
| | 2 | 2 | 2-5 | Few | Upper level |
| | | | 5-10 | Relatively few | Medium level |
| | | | 10-25 | Relatively few | Lower level |
| Low density | 1 | 1 | 1-2 | Quite few | Upper level |
| | | | 2-5 | Few | Medium level |
| | | | 5-10 | Relatively few | Lower level |
| | T | 0.5 | <1 | Occasional | Upper level |
| | | | 1-2 | Quite few | Medium level |
| | | | 2-5 | Few | Lower level |
| Extremely | 0 | 0.1 | <0.1 | 1-3 plants | Upper level |
| low density | | | <1 | Occasional | Medium level |
| | | | <2 | Quite few | Lower level |

(3) During a weed survey in the growth stage, a “W”-shaped nine-point quadrat method is used, types of weeds and a quantity of plants of each type of weed in each of quadrats with an area of 0.5 m*0.5 m are counted, and a weed density is calculated.

(4) After crop harvest (or before crop planting), a soil weed seed bank is surveyed: 9 sampling points are designed in each field according to a “W” shape, and 5 points are set around the sampling points with an interval of one meter according to a shape of “plum blossom”; one soil sample is drilled at each point, a drilling depth is 15 cm, a drilling area of each soil sample is 0.002 m² (a drilling diameter is 50 mm); and after the soil sample is treated by a water washing method, types and quantities of weed seeds in the seed bank are counted as a soil weed seed bank scale (or density).

After a rapid and quantitative survey for weed hazard in the field, a weed prevention and control policy is selected according to occurrence of weed seedlings, a dominance level (or a hazard level) of adult plants, a weed density, or a soil weed seed bank scale (or density). Refer to Table 3 for specific selection of weed control measures.

TABLE 3
Selection of weed prevention and control policy

| Survey value | | | | |
|---|---|---|---|---|
| | | | Seed bank | |
| Seedlings | Adult plants | Weed density | density | |
| (occurrence) | (hazard level) | (plants/m²) | (grains/m²) | Measure selection |
| High density | Levels 4-5 | >200 | >100000 | Conventional chemical |
| | (serious) | | | weeding + damming + net |
| | | | | fishing |
| Medium | Levels 2-3 | 100-200 | 50000-10000 | Reduced chemical weeding + |
| density | (heavy) | | | damming + net fishing |
| Low density | Level 1 (light) | 50-100 | 20000-50000 | Chemical weeding (organic |
| | | | | weeding) once + damming + |
| | | | | net fishing |
| Extremely | Level T | <50 | <20000 | Damming + net fishing |
| low density | (extremely light) | | | |

The four-level visual observation method and the seven-level visual observation method of the weeds may be applied to rapid and quantitative weed monitoring in the farming mode of rice-wheat, rice-rape, rice-green manure, or rice-winter fallow field, to guide the selection of the weed prevention and control measures. The weed occurrence (or hazard) is continuously monitored, and weed control measures are adjusted in time according to a monitoring result. 1. When the weed occurrence is of the high density, a local conventional chemical weeding manner is used in cooperation with ecological weed control measures “damming” and “net fishing”, to rapidly reduce the weed occurrence in the field. 2. When the weed occurrence is of the medium density, based on the local conventional chemical weeding manner, use of chemical herbicides (stem and leaf treatment) is reduced once in each crop season, in cooperation with the ecological weed control measures “damming” and “net fishing”, thereby effectively controlling the weed occurrence. 3. When the weed occurrence is of the low density, chemical herbicides (for soil sealing treatment or stem and leaf treatment in the seedling stage) are used only once in each crop season, in cooperation with the ecological weed control measures “damming” and “net fishing”, thereby continuously controlling the weed occurrence while reducing the usage amount (or usage frequency) of herbicides. 4. When the weed occurrence is of the extremely low density, the ecological weed control measures “damming” and “net fishing” are only implemented in crop seasons, thereby maintaining the weed occurrence at a low level.

Implementation of the ecological weed control measures “damming” and “net fishing” in different planting modes: a field is ploughed before rice planting after wheat/rape/green manure/winter fallow field, and then is irrigated. During irrigation, the measure “damming” is used, that is, before the first irrigation, a filter screen is set up at an inlet and an outlet of the field, to filter and remove weed seeds in irrigation water stream. The filter screen may be made of nylon mesh, stainless steel wire mesh or wire mesh, and an aperture of the filter screen is 50-150 mesh. The field is soaked after being irrigated. During the soaking period, a water layer of over 10 cm is maintained, and the measure “net fishing” is used, that is, when weed seeds floating on a water surface are blown to a corner or an edge of the field, a fishing net is used to remove the weed seeds floating on the water surface. The fishing net may be made of nylon mesh with an aperture of 80-120 mesh. In a late period of the soaking, the field is rotarily ploughed. After being leveled, the field is allowed to stand and maintain the water layer for a period of time, and then a “net fishing” operation is performed again.

### EMBODIMENT 1

Implementation time: 2016-2018.

Implementation location: Zhengyang Planting Specialized Cooperative Organization, Tuyuan Township, Sucheng District, Suqian City, Jiangsu Province.

Implementation solution: the test site was of a rice-wheat continuous farming mode, where wheat with a variety of “Huaimai 26” was planted in winter, with a sowing amount of 35 kg/mu; and rice with a variety of “Lianjing No. 11” was planted in summer, with a transplanting (machine transplanting) row spacing of 30 cm, a plant spacing of 20 cm, and each hole of 5 seedlings. Before test implementation: conventional chemical weeding in the test site. During wheat planting, “sealing once+stem and leaf twice” was implemented, that is, a soil sealing herbicide was used to spray before seedling after wheat sowing; during wheat of three leaves and one heart, a stem and leaf herbicide was used for spray treatment; and after wheat of four leaves to before jointing, the stem and leaf herbicide was supplementarily applied once. During rice planting, herbicides were used as “first for sealing, second for killing, and third for supplementing”. That is, the sealing herbicide was applied before seedling after rice sowing, the stem and leaf herbicide was sprayed during the seedling stage after sowing (before weeds of three leaves and one heart), and the stem and leaf herbicide was supplementarily applied once again during a late period of seedling.

Quantitative weed monitoring: a soil seed bank base survey after rice harvest in 2016. During wheat planting in 2016, a survey (a basic survey) of weed occurrence during the seedling stage of the wheat field was performed in mid-November 2016 (no herbicides were applied). After that, survey and monitoring of the above-ground weed communities in the seedling stage and the mature stage in each crop season were performed, and the selection of subsequent weed control measures was guided based on survey results, where a weed survey during the seedling stage of the rice field was performed in 30 days after rice transplantation, and a weed survey during the mature stage was performed in early October (before rice harvest); and a weed survey during the seedling stage of the wheat field was performed in 30 days after wheat sowing, and a weed survey during the mature stage was performed in early May (before wheat harvest). A soil weed seed bank survey was performed after crop (rice and wheat) harvest.

Survey results showed that the weed occurrence in the wheat field in 2016 was of medium density, that is, a small amount of green weeds covered rows when a surveyor observes when standing straightly, and weed plants can be observed when bending down, and weed seedlings can be observed after squatting down. A quantity of weed seedlings was 123 plants/m², and the soil weed seed bank was of a medium scale of 85456 grains/m². Refer to Table 4 for weed monitoring and selection of corresponding weed control measures.

TABLE 4
Weed monitoring and selection of weed control measures in test site in Suqian

| | Weed occurrence | | | | | | | |
|---|---|---|---|---|---|---|---|---|
| | (hazard level) | | | | Seed bank density | | | |
| | Mature | Seedling | | Seedling | (grains/m²) | | Selection of weed | |
| | stage of | stage of | Mature | stage of | After | | control measures | |
| | wheat | rice | stage of | wheat | wheat | After rice | | Wheat |
| Year | field | field | rice field | field | harvest | harvest | Rice field | field |
| 2016 | / | / | / | Medium | / | 85456 | Conventional | Stem and |
| | | | | density | | | chemical | leaf twice |
| | | | | 123 | | | weeding: | |
| | | | | plants/m² | | | first for | |
| | | | | | | | sealing | |
| | | | | | | | second for | |
| | | | | | | | killing, and | |
| | | | | | | | third for | |
| | | | | | | | supplement | |
| 2017 | Level 3 | Medium | Level 3 | Medium | 62023 | 64390 | ″Damming + | Sealing |
| | | density | | density | | | net fishing″ + | once + |
| | | 103 | | 108 | | | ″first for | stem and |
| | | plants/m² | | plants/m² | | | sealing, and | leaf once |
| | | | | | | | second for | |
| | | | | | | | killing″ | |
| 2018 | Level 2 | Low density | Level 2 | Medium | 49898 | 56390 | ″Damming + | Sealing |
| | | 78 plants/m² | | density | | | net fishing″ + | once + |
| | | | | 86 | | | ″first for | stem and |
| | | | | plants/m² | | | sealing, and | leaf once |
| | | | | | | | second for | |
| | | | | | | | killing″ | |

According to a survey result of weeds in the wheat field, weed control measures were guided for implementation. According to a survey result of the weed occurrence of the wheat field in 2016, the weed occurrence was of the medium density, and a comprehensive weed control measure of “reduced chemical weeding+damming+net fishing” was used to reduce the weed occurrence and the seed bank scale, that is, herbicide “sealing” treatment was not performed in a current season, “stem and leaf once” treatment was performed in the end of November (before weeds of three leaves during wheat of three leaves), and the “stem and leaf once” treatment was performed again in late February. According to a quantitative weed monitoring result in the wheat field in 2016, compared with local conventional chemical weeding, a frequency of the chemical weeding may be reduced appropriately in 2017, and through cooperation of ecological weed control measures, the comprehensive weed control measure of “reduced chemical weeding+damming+net fishing” was used to reduce the weed occurrence and the seed bank scale. That is, the measures of “damming” and “net fishing” were adopted before rice planting after wheat harvest in 2017; and the treatment of “sealing once+stem and leaf once” was implemented during rice planting, the sealing herbicide was used for “first for sealing” after the whole field was ploughed, and the stem and leaf herbicide was sprayed for “second for killing” during the seedling stage after sowing (before weeds of three leaves and one heart). Through continuous weed monitoring, the selection of weed control measures of next crop season can be properly guided. According to a continuous quantitative weed monitoring result in 2017, the weed control policy of “reduced chemical weeding+damming+net fishing” was continuously selected in 2018. The weed monitoring result showed that the weed hazard levels in the wheat and rice fields was reduced from 2016 to 2018. In general, through quantitative weed monitoring, the selection of weed control policy was more proper, and the usage frequency (or usage amount) of herbicides was reduced based on effective weed control.

### EMBODIMENT 2

Implementation time: 2016-2018.

Implementation location: Xiajia Lake Village, Lingqiao Township, Huaiyin District, Huai'an City, Jiangsu Province.

Implementation solution: the test site was of a rice-wheat continuous farming mode, where wheat with a variety of “Huaimai 26” was planted in winter, with a sowing amount of 35 kg/mu; and rice with a variety of “Lianjing No. 11” was planted in summer, with a transplanting (machine transplanting) row spacing of 30 cm, a plant spacing of 20 cm, and each hole of 5 seedlings. Before test implementation: conventional chemical weeding in the test site. During wheat planting, “sealing once+stem and leaf once” was implemented, that is, a soil sealing herbicide was used to spray before seedling after wheat sowing; and during wheat of three leaves and one heart, a stem and leaf herbicide was used for spray treatment. During rice planting, herbicides were used as “first for sealing, second for killing, and third for supplementing”. That is, the sealing herbicide was applied before seedling after rice sowing, the stem and leaf herbicide was sprayed during the seedling stage after sowing (before weeds of three leaves and one heart), and the stem and leaf herbicide was supplementarily applied once again during a late period of seedling.

Quantitative weed monitoring: during rice planting in 2016, a survey (a basic survey) of weed occurrence during the seedling stage of a rice field was performed in early July 2016 in which no herbicides were applied; and a weed survey during the mature stage of the rice field was performed in early October 2016. After that, survey and monitoring of the above-ground weed communities in the seedling stage and the mature stage in each crop season were performed, and the selection of subsequent weed control measures was guided based on survey results, where a weed survey during the seedling stage of the rice field was performed in 30 days after rice transplantation, and a weed survey during the mature stage was performed in early October (before rice harvest); and a weed survey during the seedling stage of the wheat field was performed in 30 days after wheat sowing, and a weed survey during the mature stage was performed in early May (before wheat harvest). A soil weed seed bank survey was performed after crop (rice and wheat) harvest.

A survey result showed that, the weed occurrence in the rice field in 2016 was of medium density, a quantity of weed seedlings was 79 plants/m², and the soil weed seed bank was of a medium scale of 75236 grains/m². Refer to Table 5 for weed monitoring and selection of corresponding weed control measures.

TABLE 5
Weed monitoring and selection of weed control measures in test site in Huai’an

| | Weed occurrence (hazard level) | | | | Seed bank density | | | |
|---|---|---|---|---|---|---|---|---|
| | Mature | | | Seedling | (grains/m²) | | | |
| | stage of | Seedling | Mature | stage of | After | | Selection of weed control | |
| | wheat | stage of | stage of | wheat | wheat | After rice | measures | |
| Year | field | rice field | rice field | field | harvest | harvest | Rice field | Wheat field |
| 2016 | / | Medium | Level 2 | Medium | / | 75236 | Stem and leaf | Sealing once + |
| | | density | | density | | | twice | stem and |
| | | 79 | | 103 | | | | leaf once |
| | | plants/m² | | plants/m² | | | | |
| 2017 | Level 2 | Medium | Level 1 | Medium | 48023 | 51257 | ″Damming + | Sealing once + |
| | | density | | density | | | net fishing″ + | stem and |
| | | 47 | | 69 | | | ″first for | leaf once |
| | | plants/m² | | plants/m² | | | sealing, and | |
| | | | | | | | second for | |
| | | | | | | | killing″ | |
| 2018 | Level 1 | Low | Level 1 | Medium | 40138 | 38925 | ″Damming + | Sealing once |
| | | density | | density | | | net fishing″ + | |
| | | 28 | | 52 | | | ″first for | |
| | | plants/m² | | plants/m² | | | sealing″ | |

After rice harvest in 2016, the seed bank was of a medium scale (75236 plants/m²), and the treatment of “sealing once+stem and leaf once” was performed during wheat planting in 2016 (wheat was planted in November, 2016). According to quantitative monitoring results of weed communities and seed banks in 2016 and 2017, compared with local conventional chemical weeding, a frequency of the chemical weeding may be reduced appropriately in 2017, and through cooperation of ecological weed control measures, a comprehensive weed control measure of “reduced chemical weeding+damming+net fishing” was used to reduce the weed occurrence and the seed bank scale. That is, the measures of “damming” and “net fishing” were adopted before rice planting after wheat harvest in 2017; and the treatment of “sealing once+stem and leaf once” was implemented during rice planting, the sealing herbicide was used for “first for sealing” after the whole field was ploughed, and the stem and leaf herbicide was sprayed for “second for killing” during the seedling stage after sowing (before weeds of three leaves and one heart). According to continuous monitoring results of weed communities and seed banks in 2017 and 2018, the weed occurrence in the rice field and the weed occurrence in the wheat field were lower, and seed bank scales were smaller (<50000 grains/m²). Therefore, a weed control policy of “chemical weeding once+damming+net fishing” was selected in 2018.

### EMBODIMENT 3

Implementation time: 2016-2018.

Implementation location: Datang Ecological Park, Qiandeng Township, Kunshan City, Jiangsu Province.

Implementation solution: the test site was of a rice-rape continuous farming mode, where rape with a variety of “Suyou No. 8” was planted in winter, with a row spacing of 40 cm and a plant spacing of 15 cm; and rice with a variety of “Sudao No. 5” was planted in summer, with a transplanting (machine transplanting) row spacing of 25 cm, a plant spacing of 24 cm, and each hole of 5 seedlings. Before test implementation: conventional chemical weeding in the test site. “Sealing+stem and leaf” treatment was used in a rape field, that is, a sealing herbicide was used for sealing treatment in two days before rape transplantation after rice harvest, and a stem and leaf spray herbicide was used during rape of six-eight leaves. During rice planting, herbicides were used as “first for sealing, second for killing, and third for supplementing”. That is, the sealing herbicide was applied before seedling after rice sowing, the stem and leaf herbicide was sprayed during the seedling stage after sowing (before weeds of three leaves and one heart), and the stem and leaf herbicide was supplementarily applied once again during a late period of seedling.

Quantitative weed monitoring: a soil seed bank base survey after rice harvest in 2016. During rape planting in 2016, a weed survey (a basic survey) in the rape field was performed in mid-November, 2016 (no herbicides were applied). After that, surveys and monitoring of the above-ground weed communities in the seedling stage and the mature stage in each crop season were performed: a weed survey during the seedling stage of the rice field was performed in 35 days after rice transplantation, and a weed survey during the mature stage was performed in early October (before rice harvest); and a weed survey during the seedling stage of the rape field was performed in 25 days after transplantation, and a weed survey during the mature stage was performed in early May (before rape harvest). A soil weed seed bank survey was performed after crop (rice and rape) harvest.

According to survey results of a soil seed bank after rice harvest and weed communities during the seedling stage of the wheat field in 2016, weed control measures were guided for implementation. Survey results showed that, weed occurrence in the rape field in 2016 was of high density (245 plants/m²), and a soil weed seed bank scale is larger (113285 grains/m²). Therefore, local conventional chemical weeding was maintained in a rape season in 2016, and through cooperation of ecological weed control measures during next rice planting season (in 2017), a comprehensive weed control measure of “conventional chemical weeding+damming+net fishing” was used to reduce the weed occurrence and the seed bank scale. Refer to Table 6 for weed monitoring and selection of corresponding weed control measures.

TABLE 6
Weed monitoring and selection of weed control measures in test site in Kunshan

| | Weed occurrence | | | | | | | |
|---|---|---|---|---|---|---|---|---|
| | (Hazard level) | | | | Seed bank density | | | |
| | | Rice | Rice | | (grains/m²) | | | |
| | Rape | field | field | Rape | After | | Selection of weed control | |
| | Mature | Seedling | Mature | Seedling | rape | After Rape | measures | |
| Year | stage | stage | stage | stage | harvest | harvest | Rice field | Rape field |
| 2016 | / | / | / | High | / | 113285 | Conventional | Stem and leaf |
| | | | | density | | | chemical | twice |
| | | | | 285 | | | weeding: first for | |
| | | | | plants/m² | | | sealing, second | |
| | | | | | | | for killing, and | |
| | | | | | | | third for | |
| | | | | | | | supplementing | |
| 2017 | Level | Medium | Level | Medium | 72481 | 89161 | ″Damming + net | Sealing once + |
| | 4 | density | 3 | density | | | fishing″ + ″first | stem and leaf |
| | | 149 | | 168 | | | for sealing, | once |
| | | plants/m² | | plants/m² | | | second for | |
| | | | | | | | killing, and third | |
| | | | | | | | for | |
| | | | | | | | supplementing″ | |
| 2018 | Level | Medium | Level | Medium | 55842 | 70877 | ″Damming + net | Sealing once + |
| | 3 | density | 2 | density | | | fishing″ + ″first | stem and leaf |
| | | 105 | | 112 | | | for sealing, and | once |
| | | plants/m² | | plants/m² | | | second for | |
| | | | | | | | killing″ | |

According to a monitoring result of weeds during the mature stage of the rape field in May, 2017, a hazard level of weeds was of high level 4. Therefore, conventional chemical weeding was still maintained in next rape planting season (in winter of 2017). A survey of weeds during the mature stage of the rice field in 2017 showed that a hazard level was of level 3 (medium level). Therefore, “reduced chemical weeding+damming+net fishing” was used in a rice planting season of 2018, and use of stem and leaf herbicide was reduced once. Weed monitoring was implemented, and through combined use of ecological weed control measures and chemical weeding, the hazard levels of weeds in the rape field and the rice field in 2018 were decreased, and the usage frequency of herbicides was reduced.

### EMBODIMENT 4

Implementation time: 2016-2018.

Implementation location: Shanggao County, Ji'an City, Jiangxi Province.

Implementation solution: the test site was of a rice-winter fallow mode. Rice with a variety of “Jiangzao 361” was planted by a rice machine in summer, and was cultivated in mid-May. Before test implementation: during winter fallow, an exterminative herbicide was used to perform “killing out” treatment once before weeds bear fruits; and treatment of “sealing twice+stem and leaf once” was implemented during rice planting, the sealing was performed once after the whole field was ploughed, sealing was performed again in 7-10 days after rice planting, and stem and leaf treatment was performed during weeds of three leaves and one heart after planting.

Quantitative weed monitoring: a basic survey of the above-grounded weed communities during the mature stage of the rice field was performed before rice harvest in 2016. After that, weed monitoring was performed twice during the rice planting season: a weed survey during the seedling stage of the rice field was performed in 30 days after rice transplantation, and a weed survey during the mature stage was performed in early October (before rice harvest). A soil weed seed bank survey was performed before crop (rice and wheat) harvest in every year. Refer to Table 7 for weed monitoring and selection of corresponding weed control measures.

TABLE 7
Weed monitoring and selection of weed control measures in test site in Jiangxi

| | | | Seed bank | | Selection |
|---|---|---|---|---|---|
| | Weed occurrence in rice field | | density | | of weed |
| | (hazard level) | | (grains/m²) | | control |
| | Seedling | Mature | Before rice | Winter | measures |
| Year | stage | stage | transplantation | fallow field | Rice field |
| 2016 | / | Level 4 | / | Killing out | Conventional chemical |
| | | | | | weeding: sealing |
| | | | | | twice + stem |
| | | | | | and leaf once |
| 2017 | Medium | Level 3 | 95463 | Killing out | “Damming + |
| | density 105 | | | | net fishing” + |
| | plants/m² | | | | conventional |
| | | | | | chemical weeding |
| 2018 | Medium | Level 3 | 82604 | Killing out | “Damming + |
| | density 93 | | | | net fishing” + |
| | plants/m² | | | | “sealing once” + |
| | | | | | “stem and leaf once” |

A survey result of weeds in the mature stage in 2016 showed that (in Table 7), the hazard level of weeds was of level 4, and hazard was relatively serious. Therefore, chemical weed control measures in 2017 were still maintained to local conventional chemical weeding in cooperation with ecological weed control measures of “damming+net fishing”. After one year of treatment, the hazard level of weeds in the mature stage of the rice field in 2017 was of medium level 3, and the hazard was lower than that in the previous year. Therefore, weed control measures of the rice field of next season (in 2018) were adjusted to reduce “sealing treatment” once. The weeds were continuously monitored, and the selection of weed control measures of the rice field of next season (in 2019) was guided according to a monitoring result in 2018. Quantitative weed monitoring provides a factual basis for the selection of the weed control policy.

### EMBODIMENT 5

Implementation time: 2016-2018.

Implementation location: Baihu Farmland, Lujiang County, Hefei City, Anhui Province.

Implementation solution: the test site was of a rice-green manure mode, where rice with a variety of “Huiliangyou 996” was planted in summer, with a row spacing of 35 cm, a plant spacing of 17 cm, and each hole of 4 seedlings. Astragalus sinicus was planted in 15 days before rice planting, was harvested during a blooming period (late April) of astragalus sinicus, and was returned to field after harvest. Rice was transplanted in early mid-June. Before test implementation: during rice planting, herbicides were used as “first for sealing, second for killing, and third for supplementing”, that is, a sealing herbicide was applied before seedling after rice sowing, a stem and leaf herbicide was sprayed during the seedling stage after sowing (before weeds of three leaves and one heart), and the stem and leaf herbicide was supplementarily applied once again during a late period of seedling.

Quantitative weed monitoring: a basic survey of weeds during the seedling stage of the rice field was performed in the end of June, 2016 (no herbicides were applied), with a four-level visual observation method. After that, weed monitoring was performed twice during rice planting: a weed survey during the seedling stage of the rice field was performed in 25 days after rice transplantation, and a weed survey during the mature stage was performed in early October (before rice harvest). A soil weed seed bank survey was performed before crop (rice and wheat) harvest in every year. Refer to Table 8 for weed monitoring and selection of corresponding weed control measures.

TABLE 8
Weed monitoring and selection of weed
control measures in test site in Anhui

| | | | Seed bank | Selection |
|---|---|---|---|---|
| | Weed occurrence in rice field | | density | of weed |
| | (hazard level) | | (grains/m²) | control |
| | Seedling | Mature | Before rice | measures |
| Year | stage | stage | transplantation | Rice field |
| 2016 | Medium | Level 3 | 98748 | Stem and |
| | density | | | leaf twice |
| | 123 | | | |
| | plants/m² | | | |
| 2017 | Medium | Level 3 | 72341 | “Damming + net |
| years | density | | | fishing” + |
| | 95 | | | “sealing |
| | plants/m² | | | once + stem |
| | | | | and leaf once” |
| 2018 | Low | Level 2 | 51204 | “Damming + net |
| years | density | | | fishing” + |
| | 76 | | | “sealing |
| | plants/m² | | | once + stem |
| | | | | and leaf once” |

Survey results of the seed bank and the weeds during the seedling stage in 2016 showed that (in Table 8), the soil seed bank was of a medium scale (98748 grains/m²), weed occurrence during the seedling stage of the rice field was of the medium density (123 plants/m²). After stem and leaf treatment was used twice, a survey of mature weeds in a current rice field showed that the hazard level of weeds was of level 3. Therefore, “damming+net fishing” was used in cooperation with “sealing once+stem and leaf once” during a rice planting period of next season (in 2017). Weed monitoring was continuously performed, and according to the results (a medium hazard of weeds) of quantitative weed monitoring in the seedling stage and the mature stage in 2017, selection of weed control measures in 2018 was still determined as “damming+net fishing” in cooperation with “sealing once+stem and leaf once”.

In the rice-green manure farming mode, in addition to the manner of returning astragalus sinicus to field, other green plants such as ryegrass and alfalfa may be used.

1. A quantitative weed monitoring method, comprising the following steps:
(1) during a weed survey in the seedling stage, observing abundance and coverage of weeds in the seedling stage using a four-level visual observation method, and determining a dominance level of the weeds, wherein the four-level visual observation method is specifically as follows:
level one: observing that soil is obviously covered with green when standing straightly at a position three meters away from a field, indicating high density occurrence of the weeds;
level two: observing that the soil is covered with green when standing straightly at an edge of the field or in the field, indicating medium density occurrence of the weeds;
level three: observing weed seedlings when bending down in the field, indicating low density occurrence of the weeds; and
level four: observing sporadic weed seedlings after squatting down in the field, indicating extremely low density occurrence of the weeds;
(2) during a weed survey in the mature stage, observing a height, abundance, and coverage of weeds relative to crops using a seven-level visual observation method, and determining a dominance level of the weeds, wherein the seven-level visual observation method is named in descending order of dominance levels of the weeds:
level one and level two: dominance levels 4 and 5, indicating serious weed hazard;
level three and level four: dominance levels 2 and 3, indicating medium weed hazard;
level five: a dominance level 1, indicating light weed hazard; and
level six and level seven: dominance levels 0 and T, indicating extremely light weed hazard;
(3) during a weed survey in the growth stage, using a “W”-shaped nine-point quadrat method, counting types of weeds and a quantity of plants of each type of weed in each of 9 quadrats with an area of 0.5-1 m² and designed in each field according to a “W” shape, and calculating a weed density; and
(4) after crop harvest, using a soil seed bank to drill the soil for sampling: 9 sampling points are designed in each field according to the “W” shape, and 5 points are set around the sampling points with an interval of one meter according to a shape of “plum blossom”; one soil sample is drilled at each point, a drilling depth is 15 cm, a drilling area of each soil sample is 0.002 m², and a drilling diameter is 50 mm; and after the soil sample is treated by a water washing method, types and quantities of weed seeds in the seed bank are counted as a soil weed seed bank scale.

2. The quantitative weed monitoring method according to claim 1, wherein when the monitoring survey result is the high density occurrence of the weeds, the dominance levels 4 to 5, the weed density of >200 plants/m², or a soil weed seed bank scale of >100000 grains/m², during crop planting, a local conventional chemical weeding manner is used, in cooperation with ecological weed control measures “damming” and “net fishing”.

3. The quantitative weed monitoring method according to claim 1, wherein when the monitoring survey result is the medium density occurrence of the weeds, the dominance levels 2 to 3, the weed density of 100-200 plants/m², or a soil weed seed bank scale of 50000-100000 grains/m², during crop planting, use of chemical herbicides is reduced once compared with the local conventional chemical weeding manner, in cooperation with ecological weed control measures “damming” and “net fishing”.

4. The quantitative weed monitoring method according to claim 1, wherein when the monitoring survey result is the low density occurrence of the weeds, the dominance level 1, the weed density of 50-100 plants/m², or a soil weed seed bank scale of 20000-50000 grains/m², during crop planting, chemical herbicides are only used once, in cooperation with ecological weed control measures “damming” and “net fishing”.

5. The quantitative weed monitoring method according to claim 1, wherein when the monitoring survey result is the extremely low density occurrence of the weeds, the dominance levels 0-T, the weed density of <50 plants/m², or a soil weed seed bank scale of <20000 grains/m², during crop planting, no chemical herbicides are used, and ecological weed control measures “damming” and “net fishing” are only used to maintain the weed density in the field at a low level.

6. The quantitative weed monitoring method according to claim 2, wherein the farming mode of the crops is rice-wheat, rice-rape, rice-green manure, or rice-winter fallow field.

7. The quantitative weed monitoring method according to claim 3, wherein the farming mode of the crops is rice-wheat, rice-rape, rice-green manure, or rice-winter fallow field.

8. The quantitative weed monitoring method according to claim 4, wherein the farming mode of the crops is rice-wheat, rice-rape, rice-green manure, or rice-winter fallow field.

9. The quantitative weed monitoring method according to claim 5, wherein the farming mode of the crops is rice-wheat, rice-rape, rice-green manure, or rice-winter fallow field.
